(19) United States
(12) Patent Application Publication
(10) Pub. No.: US 2012/0027816 A1
(43) Pub. Date: Feb. 2, 2012

(54) HIGHLY PURE ELETRIPTAN OR A PHARMACEUTICALLY ACCEPTABLE SALT THEREOF SUBSTANTIALLY FREE OF ELETRIPTAN N-OXIDE IMPURITY

(75) Inventors: Vijaya Gopal Kusumba, Andhra Pradesh (IN); Sampath Kumar Sankineni, Andhra Pradesh (IN); Praveen Kumar Neela, Andhra Pradesh (IN); Nitin Sharadchandra Pradhan, Maharashtra (IN)

(73) Assignee: ACTAVIS GROUP PTC EHF, Hafnarfjordur (IS)

(21) Appl. No.: 13/203,243

(22) PCT Filed: Feb. 25, 2010
(86) PCT No.: PCT/IB2010/000565

§ 371 Date: Oct. 3, 2011

(30) Foreign Application Priority Data
Feb. 25, 2009 (IN) 411/CHE/2009

(51) Int. Cl. A61K 31/404; A61K 9/14; A61P 25/30; A61P 3/04; A61P 25/24; A61P 25/06; A61P 25/00; A61P 29/00; C07D 403/06; A61P 25/22

(52) U.S. Cl. 424/400; 548/468; 514/414

(57) ABSTRACT

Provided herein is an impurity of eletriptan, eletriptan N-oxide, (R)-5-[2- (phenylsulfonypethyl]-3-[(1-methyl-2-pyrrolidinyl-N-oxide)methyl]-1H-indole, and a process for the preparation and isolation thereof. Provided further herein is a highly pure eletriptan or a pharmaceutically acceptable salt thereof substantially free of eletriptan N-oxide impurity, processes for the preparation thereof, and pharmaceutical compositions comprising highly pure eletriptan or a pharmaceutically acceptable salt thereof substantially free of eletriptan N-oxide impurity.

### CROSS REFERENCE TO RELATED APPLICATION

This application claims the benefit of priority to Indian provisional application No. 411/CHE/2009, filed on Feb. 25, 2009, which is incorporated herein by reference in its entirety.

### FIELD OF THE DISCLOSURE

Disclosed herein is an impurity of eletriptan, the N-oxide impurity, and a process for the preparation and isolation thereof. Disclosed further herein is a highly pure eletriptan or a pharmaceutically acceptable salt thereof substantially free of eletriptan N-oxide impurity, processes for the preparation thereof, and pharmaceutical compositions comprising highly pure eletriptan or a pharmaceutically acceptable salt thereof substantially free of eletriptan N-oxide impurity.

### BACKGROUND

Eletriptan, chemically named (R)-5-[2-(phenylsulfonyl)ethyl]-3-[(1-methyl-2-pyrrolidinyOmethyl]-1H-indole, is a selective 5 -hydroxytryptamine 1B/1D (5-HT1B/1D) receptor agonist and may be used in the treatment of depression, anxiety, eating disorders, obesity, drug abuse, cluster headache, migraine, chronic paroxysmal hemicrania and headache associated with vascular disorders, pain, and other disorders arising from deficient serotonergic neurotransmission. Eletriptan is represented by the following structural formula I:

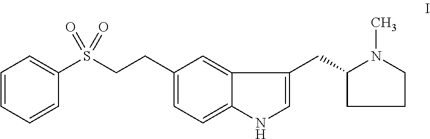

and its first synthesis was disclosed in U.S. Pat. No. 5,545,644 (hereinafter referred to as the '644 patent). Eletriptan is sold by Pfizer under the brand name RELPAX® for the treatment of migraine headaches. It is orally administered as tablets containing 24.2 mg or 48.5 mg of eletriptan hydrobromide equivalent to 20 mg or 40 mg of eletriptan. According to the process exemplified in the '644 patent, eletriptan is prepared by the reduction of (R)-5-(2-benzenesulphonylethenyl)-3-(N-methylpyrrolidin-2-ylmethyl)-1H-indole hydrobromide in the presence of 10% palladium on carbon in a solvent mixture of absolute ethanol, N,N-dimethylformamide and water, under a hydrogen atmosphere at room temperature for 18 hours, to produce a reaction mixture, followed by filtration through a Celite filter aid, and washing of the residue with ethanol. The combined filtrate and washings are evaporated under reduced pressure, and the residue is partitioned between ethyl acetate and aqueous sodium carbonate solution. The resulting organic layer is separated, washed with water and brine solution and then dried, followed by evaporation of the solvent. The resulting gum is chromatographed on silica gel eluting with dichloromethane/methanol/concentrated aqueous ammonia to yield eletriptan free base as a foam. The eletriptan free base obtained is further converted to its hemisuccinate salt by combining a solution of succinic acid in ethanol with a solution of eletriptan base in ethanol, followed by evaporation of the solvent to give eletriptan hemisuccinate as a foam.

Eletriptan obtained by the processes described in the above prior art does not have satisfactory purity for pharmaceutical use. Unacceptable amounts of impurities are generally formed along with eletriptan. In addition, the processes involve the additional step of column chromatographic purifications. Methods involving column chromatographic purifications are generally undesirable for large-scale operations as they require additional expensive setup, adding to the cost of production and thereby making the processes commercially unfeasible.

Salts of eletriptan, including the hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate salts, have been described in the literature. Eletriptan has been exemplified as a free base and hemisuccinate salt in the '644 patent. The '644 patent also discloses four solid forms (α-form, β-form, o-form and s-form) of eletriptan hydrobromide salt, compositions comprising the solid forms, methods of making the solid forms and methods of use thereof.

While the '644 patent generally mentions that the basic compounds disclosed in that patent can form a salt with pharmaceutically acceptable organic or inorganic acids such as hydrochloric acid, hydrobromic acid, hydroiodic acid, nitric acid, sulfuric acid, phosphoric acid, acetic acid, lactic acid, citric acid, tartaric acid, succinic acid, maleic acid, fumaric acid, gluconic acid, saccharic acid, benzoic acid, methanesulfonic acid and pamoic acid, only the hydrobromide and hemisuccinate salts of eletriptan have been reported as prepared and/or isolated.

U.S. Pat. No. 6,110,940 (hereinafter referred to as the '940 patent) discloses two crystalline forms (α-form and β-form) of eletriptan hydrobromide, processes for their preparation, and characterizes them by powder X-ray diffraction (P-XRD), infra-red spectroscopy (IR) and Differential Scanning Calorimetry (DSC).

The '940 patent further teaches that attempts have been made to obtain a suitable form of the following salts: hydrochloride, hydrobromide, hemisulphate, bisulphate, nitrate, acid phosphate, phosphate, methanesulphonate, benzenesulphonate, p-toluenesulphonate, (+)-camphorsulphonate, acetate, benzoate, citrate, hemifumarate, fumarate, hemimaleate, maleate, hemisuccinate, succinate, hemi-L-tartrate, L-tartrate, hemi-D-tartrate, D-tartrate, L-lactate, hippurate, (R)-(−)-mandelate, hemiphthalate, phthalate and hemiterephthalate. Of these thirty possible salts, only four could be obtained as crystalline solids, namely the hemisulphate, hydrochloride, hydrobromide and benzenesulphonate salts; the remainder were obtained as non-crystalline/low or non-sharp melting/sticky solids, gums, glasses, froths, resins or oils. Moreover, of the four crystalline salts, the benzenesulphonate salt proved to have an insufficiently high melting point (m.p.) of 74-75° C.

European Patent No. 1135381 B1 and subsequent equivalent U.S. Pat. No. 7,238,723 B2 disclose a crystalline monohydrate of eletriptan hydrobromide and processes for its preparation.

PCT Publication No. WO 2009/077858, filed by the present inventors, which is incorporated herein by reference in its entirety, discloses novel hemioxalate salt of eletriptan, solid state forms of eletriptan hemioxalate, processes for the preparation, pharmaceutical compositions, and method of treating thereof.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in eletriptan or any active pharmaceutical ingredient (API) are undesirable and might be harmful.

Regulatory authorities worldwide require that drug manufacturers isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product is analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United

States Food and Drug Administration guidelines recommend that the amounts of some impurities are limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRT”) to identify impurities. The RRT of an impurity is its retention time divided by the retention time of a reference marker.

It is known by those skilled in the art, the management of process impurities is greatly enhanced by understanding their chemical structures and synthetic pathways, and by identifying the parameters that influence the amount of impurities in the final product.

There is a need for highly pure eletriptan or a pharmaceutically acceptable salt thereof substantially free of impurities, as well as processes for preparing thereof

The specific surface area of an active pharmaceutical ingredient may be affected by various factors. There is a general connection between Specific Surface Area and Particle Size; the smaller the Particle Size, the higher the Specific Surface Area. The rate of dissolution of a poorly-soluble drug is a rate-limiting factor in its absorption by the body. A reduction in the particle size can increase the dissolution rate of such compounds through an increase in the surface area of the solid phase that is in contact with the liquid medium, thereby resulting in an enhanced bioavailability of the compositions containing such compounds. It is generally not possible to predict the exact particle size and distribution required for any particular drug substance to achieve a specific dissolution profile or a specific in vivo behavior, as different drugs show differing dissolution characteristics with a reduction in the particle size.

There is a need in the art for highly pure eletriptan or a pharmaceutically acceptable salt thereof, with reduced particle size distribution, which has good flow properties, and better dissolution and solubility properties to obtain formulations with greater bioavailability.

### SUMMARY

In one aspect, provided herein is an isolated eletriptan N-oxide compound, (R)-5-[2-(phenylsulfonyl)ethyl]-3-[(1-methyl-2-pyrrolidinyl-N-oxide)methyl]-1H-indole, having the following structural formula A:

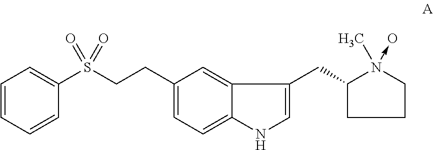

or a pharmaceutically acceptable acid addition salt thereof. The compound of formula A is also referred to herein as the eletriptan N-oxide impurity.

In another aspect, provided herein is a highly pure eletriptan or a pharmaceutically acceptable salt thereof substantially free of eletriptan N-oxide impurity.

In yet another aspect, encompassed herein is a process for preparing the highly pure eletriptan or a pharmaceutically acceptable salt thereof substantially free of eletriptan N-oxide impurity.

In another aspect, provided herein is a pharmaceutical composition comprising highly pure eletriptan or a pharmaceutically acceptable salt thereof substantially free of eletriptan N-oxide impurity, and one or more pharmaceutically acceptable excipients.

In still another aspect, provided herein is a pharmaceutical composition comprising highly pure eletriptan or a pharmaceutically acceptable salt thereof substantially free of eletriptan N-oxide impurity made by the process disclosed herein, and one or more pharmaceutically acceptable excipients.

In still further aspect, encompassed is a process for preparing a pharmaceutical formulation comprising combining highly pure eletriptan or a pharmaceutically acceptable salt thereof substantially free of eletriptan N-oxide impurity with one or more pharmaceutically acceptable excipients.

In another aspect, the highly pure eletriptan or a pharmaceutically acceptable salt thereof substantially free of eletriptan N-oxide impurity disclosed herein for use in the pharmaceutical compositions has a D₉₀ particle size of less than or equal to about 200 microns, specifically about 1 micron to about 150 micron, and most specifically about 10 microns to about 100 microns.

### DETAILED DESCRIPTION

According to one aspect, there is provided an eletriptan N-oxide compound, (R)-5-[2-(phenylsulfonyl)ethyl]-3-[(1-methyl-2-pyrrolidinyl-N-oxide)methyl]-1H-indole, having the following structural formula A:

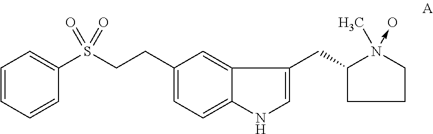

or a pharmaceutically acceptable acid addition salt thereof.

The acid addition salts of eletriptan N-oxide can be derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, benzenesulfonic acid, toluenesulfonic acid, citric acid, and tartaric acid.

Specific pharmaceutically acceptable acid addition salts of eletriptan N-oxide are hydrochloride, hydrobromide, hemioxalate, hemisulphate, hemifumarate, fumarate, hemisuccinate, succinate, maleate, fumarate, besylate, tosylate, tartrate; and more specifically hydrobromide and hemioxalate.

According to another aspect, there is provided an impurity of eletriptan, the eletriptan N-oxide impurity, (R)-5-[2-(phenylsulfonyl)ethyl]-3-[(1-methyl-2-pyrrolidinyl-N-oxide)methyl]-1H-indole, of formula A.

The eletriptan N-oxide impurity has been identified, isolated and synthesized. The eletriptan N-oxide impurity was detected and resolved from eletriptan by HPLC with an RRt of 0.72. The structure of the compound of formula A was deduced with the aid of ¹H, ¹³C NMR and IR spectroscopy and FAB mass spectrometry. The parent ion at 399 is consistent with the assigned structure.

According to another aspect, there is provided an isolated eletriptan N-oxide impurity. Eletriptan N-oxide formed during the synthesis of eletriptan or a pharmaceutically acceptable salt thereof can be isolated by subjecting the eletriptan or a pharmaceutically acceptable salt thereof that contains the eletriptan N-oxide impurity to column chromatography. The column chromatography comprises using a silica gel, as a stationary phase, and a gradient of eluents that remove eletriptan N-oxide impurity from the column on which it adsorbed.

In one embodiment, the eletriptan N-oxide of formula A is prepared according to the process exemplified in the Example 3 as disclosed herein.

Regarding the specific RRt value of impurity disclosed herein, it is well known to a person skilled in the art that the RRt values may vary from sample to sample due to, inter alia, instrument errors (both instrument to instrument variation and the calibration of an individual instrument) and differences in sample preparation. Thus, it has been generally accepted by those skilled in the art that independent measurement of an identical RRt value can differ by amounts of up to ±0.02.

Thus there is a need for a method for determining the level of impurities in eletriptan samples and removing the impurities.

Extensive experimentation was carried out by the present inventors to reduce the level of the eletriptan N-oxide impurity in eletriptan. As a result, it has been found that the eletriptan N-oxide impurity formed in the preparation of the eletriptan can be reduced or completely removed by the processes disclosed herein.

According to another aspect, there is provided a highly pure eletriptan or a pharmaceutically acceptable salt thereof substantially free of eletriptan N-oxide impurity.

As used herein, “highly pure eletriptan or a pharmaceutically acceptable salt thereof substantially free of eletriptan N-oxide impurity” refers to eletriptan or a pharmaceutically acceptable salt thereof comprising the eletriptan N-oxide impurity in an amount of less than about 0.25 area-% as measured by HPLC. Specifically, the eletriptan, as disclosed herein, contains less than about 0.15 area-%, more specifically less than about 0.05 area-%, still more specifically less than about 0.02 area-% of the eletriptan N-oxide impurity, and most specifically is essentially free of the eletriptan N-oxide impurity.

In one embodiment, the highly pure eletriptan or a pharmaceutically acceptable salt thereof disclosed herein comprises the eletriptan N-oxide impurity in an amount of about 0.01 area-% to about 0.15 area-%, specifically in an amount of about 0.01 area-% to about 0.05 area-%, as measured by HPLC.

In another embodiment, the highly pure eletriptan or a pharmaceutically acceptable salt thereof disclosed herein has a purity of greater than about 99%, specifically greater than about 99.5%, more specifically greater than about 99.9%, and most specifically greater than about 99.95% as measured by HPLC. For example, the purity of the highly pure eletriptan or a pharmaceutically acceptable salt thereof is about 99% to about 99.95%, or about 99.5% to about 99.99%.

In yet another embodiment, the highly pure eletriptan or a pharmaceutically acceptable salt thereof disclosed herein is essentially free of the eletriptan N-oxide impurity.

The term “eletriptan or a pharmaceutically acceptable salt thereof essentially free of eletriptan N-oxide impurity” refers to eletriptan or a pharmaceutically acceptable salt thereof contains a non-detectable amount of the eletriptan N-oxide impurity as measured by HPLC.

According to another aspect, there is provided a process for preparing highly pure eletriptan or a pharmaceutically acceptable salt thereof substantially free of eletriptan N-oxide impurity, comprising:

a) contacting crude eletriptan free base with oxalic acid in a first solvent to produce a first reaction mass containing eletriptan hemioxalate salt;
b) optionally, heating the first reaction mass obtained in step-(a);
c) substantially removing the first solvent from the first reaction mass obtained in step-(a) or step-(b) to produce a solid form of highly pure eletriptan hemioxalate substantially free of eletriptan N-oxide impurity; or
d) isolating the solid form of highly pure eletriptan hemioxalate substantially free of eletriptan N-oxide impurity from the first reaction mass obtained in step-(a) or step-(b) by forcible or spontaneous crystallization;
e) reacting the highly pure eletriptan hemioxalate obtained in step-(c) or step-(d) with an acid and/or a base in a second solvent to produce a second reaction mass containing eletriptan or a pharmaceutically acceptable salt thereof substantially free of eletriptan N-oxide impurity; and
f) isolating and/or recovering the highly pure eletriptan or a pharmaceutically acceptable salt thereof substantially free of eletriptan N-oxide impurity from the second reaction mass.

Exemplary pharmaceutically acceptable salts of eletriptan include, but are not limited to, hydrochloride, hydrobromide, hemioxalate, hemisulphate, phosphate, hemifumarate, fumarate, hemisuccinate, succinate, maleate, fumarate, besylate, tosylate and tartrate. Specific pharmaceutically acceptable salts of eletriptan are hydrobromide and hemioxalate.

Exemplary first solvents used in step-(a) include, but are not limited to, water, an alcohol, a ketone, a chlorinated hydrocarbon, a hydrocarbon, a nitrile, an ester, an ether, a polar aprotic solvent, and mixtures thereof The term solvent also includes mixtures of solvents.

In one embodiment, the first solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, N,N-dimethylformamide, N,N-dimethylacetamide, dimethylsulfoxide, and mixtures thereof; more specifically, the first solvent is selected from the group consisting of water, methanol, ethanol, isopropanol, acetonitrile, and mixtures thereof; and most specifically methanol.

In one embodiment, the reaction in step-(a) is carried out at a temperature of about 0° C. to the reflux temperature of the solvent used, specifically at about 0° C. to about 80° C., and more specifically at about 20° C. to about 60° C.

In another embodiment, the reaction mass in step-(b) is heated at a temperature of about 40° C. to the reflux temperature of the solvent used for at least 20 minutes, and more specifically at the reflux temperature of the solvent used for about 30 minutes to about 5 hours.

The term “substantially removing” the solvent refers to at least 60%, specifically grater than about 85%, more specifically grater than about 90%, still more specifically grater than about 99%, and most specifically essentially complete (100%), removal of the solvent from the solvent solution.

Removal of solvent in step-(c) is accomplished, for example, by substantially complete evaporation of the solvent, concentrating the solution or distillation of solvent, under inert atmosphere.

In one embodiment, the solvent is removed by evaporation. Evaporation can be achieved at sub-zero temperatures by lyophilization or freeze-drying techniques. The solution may also be completely evaporated in, for example, a pilot plant Rota vapor, a

Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Another suitable method is vertical agitated thin-film drying (or evaporation). Agitated thin film evaporation technology involves separating the volatile component using indirect heat transfer coupled with mechanical agitation of the flowing film under controlled conditions. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution or a combination thereof

The term “anti-solvent” refers to a solvent which when added to an existing solution of a substance reduces the solubility of the substance.

Exemplary anti-solvents include, but are not limited to, an ether, a hydrocarbon solvent, and mixtures thereof

In one embodiment, the anti-solvent is selected from the group consisting of diisopropyl ether, diethyl ether, tetrahydrofuran, dioxane, n-pentane, n-hexane and n-heptane and their isomers, cyclohexane, toluene, xylene, and mixtures thereof Specific anti-solvents are diisopropyl ether, diethyl ether and mixtures thereof In one embodiment, the crystallization is carried out by stirring the reaction mass at a temperature of below 35° C., specifically at a temperature of about 0° C. to about 30° C., and most specifically at about 20° C. to about 30° C.

The highly pure eletriptan hemioxalate substantially free of eletriptan N-oxide impurity obtained in step-(d) is recovered by methods such as filtration, filtration under vacuum, decantation, centrifugation, or a combination thereof In one embodiment, the highly pure eletriptan hemioxalate substantially free of eletriptan N-oxide impurity is recovered by filtration employing a filtration media of, for example, a silica gel or celite.

The highly pure eletriptan hemioxalate substantially free of eletriptan N-oxide impurity obtained by the above process may be further dried in, for example, a Vacuum Tray Dryer, a Rotocon Vacuum Dryer, a Vacuum Paddle Dryer or a pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

The total purity of the eletriptan hemioxalate salt obtained by the process disclosed herein is of greater than about 99%, specifically greater than about 99.5%, and more specifically greater than about 99.95% as measured by HPLC.

Exemplary second solvents used in step-(e) include, but are not limited to, water, an alcohol, a ketone, a nitrile, an ester, an ether, a polar aprotic solvent, and mixtures thereof The term solvent also includes mixtures of solvents.

In one embodiment, the second solvent is selected from the group consisting of water, methanol, ethanol, n-propanol, isopropanol, n-butanol, isobutanol, tert-butanol, amyl alcohol, acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, tetrahydrofuran, dioxane, diethyl ether, diisopropyl ether, monoglyme, diglyme, N,N-dimethylformamide, N,N-dimethylacetamide, dimethylsulfoxide, and mixtures thereof; more specifically, the second solvent is selected from the group consisting of water, methanol, ethanol, isopropanol, n-butanol, and mixtures thereof; and most specifically isopropanol. The base used in step-(e) is an organic or inorganic base. Specific organic bases are triethylamine, tributylamine, diisopropylethylamine, diethylamine, tert-butyl amine, N-methylmorpholine, pyridine, 4-(N,N-dimethylamino)pyridine, and mixtures thereof Exemplary inorganic bases include, but are not limited to, ammonia; hydroxides, alkoxides, carbonates and bicarbonates of alkali or alkaline earth metals. Specific inorganic bases are ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide, potassium tert-butoxide, and mixtures thereof; and more specifically ammonia, sodium bicarbonate, sodium hydroxide, potassium hydroxide, sodium carbonate and potassium carbonate.

If the reaction in step-(e) is carried out in the presence of a base the product obtained is eletriptan base, which can be in-situ converted into a pharmaceutically acceptable acid addition salt of eletriptan using a suitable acid in a suitable solvent. In one embodiment, the pharmaceutically acceptable acid addition salts of eletriptan can be obtained directly in step-(e) by carrying out the reaction in the presence of a suitable acid.

Exemplary acids used in step-(e) include, but are not limited to, organic and inorganic acids, for example, hydrochloric acid, hydrobromic acid, hydroiodic acid, acetic acid, fumaric acid, maleic acid, tartaric acid, di-p-toluoyl-L-(+)-tartaric acid, succinic acid, benzenesulfonic acid, toluenesulfonic acid, methanesulfonic acid, and a most specific acid is hydrobromic acid.

In one embodiment, the hydrobromic acid used may be in the form of concentrated hydrobromic acid or aqueous hydrobromic acid or in the form of hydrobromic acid diluted in an organic solvent. The solvent used for diluting hydrobromic acid is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof

In one embodiment, the reaction in step-(e) is carried out at a temperature of about −25° C. to the reflux temperature of the solvent used, specifically at about 0° C. to about 80° C. and more specifically at about 20° C. to about 60° C.

In another embodiment, the hydrobromic acid is used in the molar ratio of about 0.8 to 1.2 moles, specifically about 0.9 to 1.0 moles, per 1 mole of the eletriptan hemioxalate in order to ensure a proper course of the reaction.

The reaction mass containing the pure eletriptan or a pharmaceutically acceptable salt thereof substantially free of eletriptan N-oxide impurity obtained in step-(e) may be subjected to usual work up such as a washing, an extraction, a pH adjustment, an evaporation or a combination thereof.

The isolation of highly pure eletriptan or a pharmaceutically acceptable salt thereof substantially free of eletriptan N-oxide impurity in step-(f) is carried out by forcible or spontaneous crystallization methods as described above.

In one embodiment, the crystallization is carried out by stirring the solution at a temperature of below 30° C. for at least 15 minutes, specifically at about 0° C. to about 30° C. for about 30 minutes to about 20 hours, and more specifically at about 0° C. to about 25° C. for about 1 hour to about 5 hours.

The recovery of pure eletriptan or a pharmaceutically acceptable salt thereof substantially free of eletriptan N-oxide impurity in step-(f) is carried out by the techniques as described hereinabove.

The highly pure eletriptan or a pharmaceutically acceptable salt thereof substantially free of eletriptan N-oxide impurity obtained by the above process may be further dried by the techniques as described hereinabove.

According to another aspect, there is provided a process for the preparation of highly pure eletriptan hydrobromide substantially free of eletriptan N-oxide impurity, comprising:

a) providing a solution or suspension of eletriptan hemioxalate in a solvent;
b) combining the solution or suspension obtained in step-(a) with hydrobromic acid; and
c) optionally, heating the reaction mass obtained in step-(b); and
d) isolating and/or recovering highly pure eletriptan hydrobromide substantially free of eletriptan N-oxide impurity from the reaction mass.

In one embodiment, the solvent used in step-(a) is selected from the group consisting of water, an alcohol, a ketone, an ester, and mixtures thereof

Specifically, the solvent is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof; more specifically, the solvent is selected from the group consisting of water, acetone, methanol, ethanol, n-propanol, isopropanol, n-butanol, isobutanol, tert-butanol, amyl alcohol, acetone, ethyl acetate, and mixtures thereof; and most specifically, the solvent is selected from the group consisting of water, acetone, methanol, ethanol, isopropanol, ethyl acetate, and mixtures thereof.

Step-(a) of providing a solution of eletriptan hemioxalate includes dissolving any form of eletriptan hemioxalate in the solvent, or obtaining an existing solution from a previous processing step.

In one embodiment, the eletriptan hemioxalate is dissolved in the solvent at a temperature of about 0° C. to about the reflux temperature of the solvent used, specifically at about 20° C. to about 100° C., and most specifically at about 25° C. to about 80° C.

Step-(a) of providing a suspension of eletriptan hemioxalate includes suspending any form of eletriptan hemioxalate in the solvent while stirring. In one embodiment, the suspension is stirred at below boiling temperature of the solvent used for at least 30 minutes and more specifically at about 25° C. to about 80° C. for about 1 hour to about 10 hours.

In yet another embodiment, the solution or suspension in step-(a) is prepared by admixing crude eletriptan base, oxalic acid and the solvent to obtain a mixture; and stirring the mixture to obtain a solution or suspension of eletriptan hemioxalate.

In one embodiment, the hydrobromic acid in step-(b) is used in the form as described above.

In another embodiment, the hydrobromic acid is used in the molar ratio as described above.

Combining of the solution or suspension with hydrobromic acid in step-(b) is done in a suitable order, for example, the solution or suspension is added to the hydrobromic acid, or alternatively, the hydrobromic acid is added to the solution or suspension. The addition is carried out drop wise, in one portion, or in more than one portion. In one embodiment, addition is carried out at a temperature of below about 60° C. for at least 15 minutes, and more specifically at a temperature of about 15° C. to about 35° C. for about 20 minutes to about 2 hours. After completion of the addition process, the resulting mass is stirred for at least 20 minutes, more specifically about 30 minutes to about 10 hours, at a temperature of about 20° C. to about 35° C.

The heating in step-(c) is carried out at a temperature of about 40° C. to the reflux temperature of the solvent used for at least 20 minutes, and more specifically at a temperature of about 40° C. to about 80° C. for about 30 minutes to about 4 hours. The solution obtained in step-(b) or step-(c) is optionally subjected to carbon treatment or silica gel treatment. The carbon treatment or silica gel treatment is carried out by methods known in the art, for example by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing eletriptan hydrobromide by removing charcoal or silica gel. Preferably, finely powdered carbon is an active carbon. A specific mesh size of silica gel is 40-500 mesh, and more specifically 60-120 mesh.

The isolation and recovery of highly pure eletriptan hydrobromide substantially free of eletriptan N-oxide impurity in step-(d) is carried out by the methods as described above.

In one embodiment, the isolation is carried out by stirring the solution at a temperature of below 35° C. for at least 20 minutes, and more specifically at about 0° C. to about 30° C. for about 1 hour to about 10 hours.

The highly pure eletriptan hydrobromide substantially free of eletriptan N-oxide impurity obtained by above process may be further dried by the methods as described above.

According to another aspect, there is provided a process for synthesizing and isolating the eletriptan N-oxide of formula A or a pharmaceutically acceptable acid addition salt thereof, comprising:

a) reacting eletriptan base with an oxidizing agent in a suitable solvent to produce a reaction mass containing eletriptan N-oxide; and
b) isolating and/or recovering the eletriptan N-oxide from the reaction mass obtained in step-(a) and optionally converting the eletriptan N-oxide obtained into a pharmaceutically acceptable acid addition salt thereof

Exemplary oxidizing agents used in step-(a) include, but are not limited to, m-chloroperbenzoic acid, hydrogen peroxide, sodium hypochlorite, cumene hydrogen peroxide, and the like. A most specific oxidizing agent is m-chloroperbenzoic acid.

Exemplary solvents used in step-(a) include, but are not limited to, a ketone, a chlorinated hydrocarbon, a hydrocarbon, a nitrile, an ester, an ether, and mixtures thereof The term solvent also includes mixtures of solvents.

In one embodiment, the solvent is selected from the group consisting of acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, and mixtures thereof; more specifically, the solvent is selected from the group consisting of hexane, heptane, cyclohexane, toluene, dichloromethane, and mixtures thereof.

In one embodiment, the reaction in step-(a) is carried out at a temperature of below about 50° C. for at least 30 minutes, specifically at a temperature of about −20° C. to about 30° C. for about 30 minutes to about 10 hours, and most specifically at about 0° C. to about 10° C. for about 1 hour to about 5 hours. In another embodiment, the reaction mass may be quenched with water after completion of the reaction.

In another embodiment, the oxidizing agent is used in a molar ratio of about 1 to 4 moles, specifically about 2 to 3 moles, per 1 mole of eletriptan free base is used in order to ensure a proper course of the reaction.

The reaction mass containing the eletriptan N-oxide obtained in step-(a) is subjected to usual work up such as a washing, a filtration, an extraction, a pH adjustment, an evaporation or a combination thereof.

In one embodiment, the isolation in step-(b) is carried by the methods such as cooling, seeding, partial removal of the solvent from the solution, by adding an anti-solvent to the solution, evaporation, vacuum drying, spray drying, freeze drying, column chromatography, or a combination thereof.

The solvent used for isolating the eletriptan N-oxide in step-(b) is selected from the group consisting of water, an alcohol, a hydrocarbon, a ketone, an ether, a nitrile, and mixtures thereof Specific solvents are water, hexane, heptane, cyclohexane, toluene, methylene chloride, acetone, and mixtures thereof.

Pharmaceutically acceptable acid addition salts of eletriptan N-oxide can be prepared by using the eletriptan N-oxide obtained by the method disclosed herein, by known methods.

Specific pharmaceutically acceptable acid addition salts of eletriptan N-oxide include, but are not limited to, hydrochloride, hydrobromide, hemioxalate, hemisulphate, phosphate, hemifumarate, fumarate, hemisuccinate, succinate, maleate, fumarate, besylate, tosylate, and tartrate.

Further encompassed herein is the use of the highly pure eletriptan or a pharmaceutically acceptable salt thereof substantially free of eletriptan N-oxide impurity for the manufacture of a pharmaceutical composition together with a pharmaceutically acceptable carrier.

A specific pharmaceutical composition of highly pure eletriptan or a pharmaceutically acceptable salt thereof substantially free of eletriptan N-oxide impurity is selected from a solid dosage form and an oral suspension.

In one embodiment, the highly pure eletriptan or a pharmaceutically acceptable salt thereof substantially free of eletriptan N-oxide impurity has a D₉₀ particle size of less than or equal to about 200 microns, specifically about 1 micron to about 150 micron, and most specifically about 10 microns to about 100 microns.

In another embodiment, the particle sizes of the highly pure eletriptan or a pharmaceutically acceptable salt thereof substantially free of eletriptan N-oxide impurity are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from depression, anxiety, eating disorders, obesity, drug abuse, cluster headache, migraine, chronic paroxysmal hemicrania and headache associated with vascular disorders, pain, and other disorders arising from deficient serotonergic neurotransmission, comprising administering a therapeutically effective amount of the highly pure eletriptan or a pharmaceutically acceptable salt thereof substantially free of eletriptan N-oxide impurity, or a pharmaceutical composition that comprises a therapeutically effective amount of highly pure eletriptan or a pharmaceutically acceptable salt thereof substantially free of eletriptan N-oxide impurity along with pharmaceutically acceptable excipients.

According to another aspect, there are provided pharmaceutical compositions comprising highly pure eletriptan or a pharmaceutically acceptable salt thereof substantially free of eletriptan N-oxide impurity prepared according to the processes disclosed herein and one or more pharmaceutically acceptable excipients.

According to another aspect, there is provided a process for preparing a pharmaceutical formulation comprising combining highly pure eletriptan or a pharmaceutically acceptable salt thereof substantially free of eletriptan N-oxide impurity prepared according to processes disclosed herein, with one or more pharmaceutically acceptable excipients.

Yet in another embodiment, pharmaceutical compositions comprise at least a therapeutically effective amount of highly pure eletriptan or a pharmaceutically acceptable salt thereof substantially free of eletriptan N-oxide impurity. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure eletriptan or a pharmaceutically acceptable salt thereof substantially free of eletriptan N-oxide impurity may also be administered as suppositories, ophthalmic ointments and suspensions, and parenteral suspensions, which are administered by other routes.

The pharmaceutical compositions further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure eletriptan or a pharmaceutically acceptable salt thereof substantially free of eletriptan N-oxide impurity within a capsule which may be coated with gelatin. Tablets and powders may also be coated with an enteric coating. Suitable enteric coating agents include phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

### EXPERIMENTAL DETAILS:

HPLC method for measuring chemical purity:

### Column: Zorbax Extend C 18 (150×4.6 mm, 5 μ)

### Make: Agilent, Part No: 773450-902

### Detector: UV at 225 nm

Flow rate: 1.0 mL/min
Injection volume: 10.0 μL
Column temp: 40° C.
Sample Conc.: 1.0 mg/mL
Diluent: Prepare a premixed mixture of water and acetonitrile in ratio of 10:90 (v/v)
Buffer preparation:
Transfer about 2 mL of Triethylamine in 1000 mL of water and adjust the pH to 7.50±0.05 with diluted orthophosphoric acid. Filter through 0.45 μm or finer porosity membrane and degas.

### Mobile Phase-A: Buffer

### Mobile Phase-B: Acetonitrile: MeOH (30:70)

Gradient programme:

| Time (min) | (%) Mobile phase-A | (%) Mobile phase-B |
|---|---|---|
| 0 | 60 | 40 |
| 40 | 20 | 80 |
| 50 | 20 | 80 |
| 51 | 60 | 40 |
| 60 | 60 | 40 |

The following examples are given for the purpose of illustrating the present invention and should not be considered as limitation on the scope or spirit of the invention.

### EXAMPLES

### Example 1

Preparation of eletriptan hemioxalate
(R)-5-[2-(Phenylsulfonyl)ethenyl]-3-[(1-methyl-2-pyrrolidinyl)methyl]-1H-indole (24 g) was taken in acetone (200 ml) and water (60 ml). Methanesulfonic acid (6.3 g) and 10% Pd/C (6 g) were added to the resulting mixture, followed by hydrogenation under a pressure of 12-15 kg/cm² for 6 hours. Acetone was distilled from the reaction mass after completion of the reaction, and the resulting mass was washed with methylene dichloride (3×75 ml). The resulting aqueous layer was collected and the pH was adjusted to 10.5−11 with a 40% sodium hydroxide solution, followed by extracting with methylene chloride (3×100 ml) to afford eletriptan base as an oil. The resulting oil was added to methanol (50 ml) at 25-30° C., followed by the drop wise addition of a solution of oxalic acid (3 g, 0.023 moles) dissolved in methanol (15 ml) at 25-30° C. over a period of 30-60 minutes. The pH of the resulting solution was adjusted to 6-7 by adding a solution of oxalic acid in methanol. The reaction mixture was stirred for 60 minutes at 25-30° C. The resulting solid was filtered and washed with methanol (15 ml) and further dried in air oven at 50-55° C. to give 15 g of eletriptan hemioxalate as a crystalline solid (HPLC purity: 99.8%; content of eletriptan N-oxide impurity: Below detection level).

### Example 2

Preparation of eletriptan hydrobromide
Eletriptan hemioxalate (20 g, 0.044 moles) was taken in isopropanol (100 ml) at 25-30° C., followed by the drop wise addition of a solution of 47% aqueous hydrobromic acid (9.3 g, 0.037 moles) diluted in isopropanol (50 ml) at 25-30° C. for 30-60 minutes. The reaction mixture was stirred for 2 hours at 25-30° C. The resulting solid was filtered and washed with isopropanol (50 ml). The resulting product was finally washed with acetone (60 ml) and further dried under vacuum at 50-55° C. to give 20 g of eletriptan hydrobromide (HPLC Purity: 99.9%; content of eletriptan N-oxide impurity: Below detection level).

### Example 3

Preparation of (R)-5-[2-(Phenylsulfonyl)ethyl]-3-[(1-methyl-2-pyrrolidinyl-N-oxide)methyl]-1H-indole (eletriptan N-oxide)
Eletriptan base (2 g) was added to methylene chloride (10 ml), and the resulting mass was cooled to 0° C., followed by slow addition of a solution of m-chloroperbenzoic acid (1.8 g) in methylene chloride (10 ml) at 0-5° C. The resulting mixture was stirred for 2 hours at 0-5° C. The resulting mass was diluted with water (10 ml) and stirred for 10 minutes at 25-27° C. The resulting organic layer was separated and washed with aqueous saturated sodium bicarbonate (3×10 ml). The organic layer was dried over sodium sulphate (1 g), followed by distillation and then subjected to column chromatography to produce eletriptan N-oxide (Purity by HPLC: 96.27%).

### Example 4

Preparation of eletriptan hydrobromide
Eletriptan hemioxalate (20 g) was taken in isopropanol (200 ml) at 25-30° C., followed by the drop wise addition of a solution of 47% aqueous hydrobromic acid (11.9 g) diluted in isopropanol (40 ml) at 25-30° C. for 60 minutes. The reaction mixture was stirred for 3 to 4 hours at 25-30° C. The resulting solid was filtered and washed with isopropanol (40 ml). The resulting product was finally washed with acetone (40 ml). Acetone (200 ml) was added to the resulting wet compound at 25-30° C., followed by heating to reflux and maintaining for 3 to 4 hours. The resulting mass was cooled to 25-30° C. under nitrogen atmosphere and maintained for 60 minutes at 25-30° C. The separated solid was filtered, washed with acetone (40 ml) at and then dried the product at 55-60° C. under vacuum for 10-12 hours to give 15.3 g of eletriptan hydrobromide (HPLC Purity: 99.9%) [Particle size distribution: d(0.1)=12.07 microns, d(0.5) =27.69 microns, d(0.9) =53.92 (Example 4A in Table 1)]. The particle size distributions of 2 additional samples, obtained according to the procedure described in example 4, are detailed in Table 1 (Examples 4B and 4C).

TABLE 1
Particle Size Distribution

| Example | d(0.1) microns | d(0.5) microns | d(0.9) microns |
|---|---|---|---|
| 4A | 12.07 | 27.69 | 53.92 |
| 4B | 9.99 | 20.85 | 42.83 |
| 4C | 13.58 | 27.25 | 50.91 |

### Example 5

Eletriptan hydrobromide (obtained from Example 4) was fine-milled by being passed through a grinder (Make: Morphy Richards, Model-Icon DLX) having stainless steel liquidizing blade for 3-4 minutes to where 90 volume-% of the eletriptan hydrobromide particles had a desired particle size.

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC(™) F68, PLURONIC(™) F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel(™)), carsium (e.g., Amberlite(™)), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN(™)s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP).

The term “crude eletriptan or a pharmaceutically acceptable salt thereof” as used herein refers to eletriptan or a pharmaceutically acceptable salt thereof containing greater than about 0.25 area-%, more specifically greater than about 0.3 area-%, still more specifically greater than about 0.4 area-%, of the eletriptan N-oxide impurity.

As used herein, the term, “detectable” refers to a measurable quantity measured using an HPLC method having a detection limit of 0.01 area-%.

As used herein, in connection with amount of impurities in eletriptan or a pharmaceutically acceptable salt thereof, the term “not detectable” means not detected by the herein described HPLC method having a detection limit for impurities of 0.01 area-%.

As used herein, “limit of detection (LOD)” refers to the lowest concentration of analyte that can be clearly detected above the base line signal, is estimated is three times the signal to noise ratio.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (PSD)” means the cumulative volume size distribution of equivalent spherical diameters as determined by laser diffraction in Malvern Master Sizer 2000 equipment or its equivalent using a particle size method containing

Tegiloxan medium with sonication time of 10 seconds and stirrer speed of 2500 rpm. “Mean particle size distribution, i.e., (D₅₀)” correspondingly, means the median of said particle size distribution.

The important characteristics of the PSD are the (D₉₀), which is the size, in microns, below which 90% of the particles by volume are found, and the (D₅₀), which is the size, in microns, below which 50% of the particles by volume are found. Thus, a D₉₀ or d(0.9) of less than 300 microns means that 90 volume-percent of the particles in a composition have a diameter less than 300 microns.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Eletriptan or a pharmaceutically acceptable salt thereof comprising a (R)-5-[2-(phenylsulfonyl)ethyl]-3-[(1-methyl-2-pyrrolidinyl-N-oxide)methyl]-1H-indole (eletriptan N-oxide impurity) in an amount of less than about 0.25 area-% as measured by HPLC, wherein the eletriptan has a purity of about 99% to about 99.99% as measured by HPLC.

2. (canceled)

3. Eletriptan of claim 1, comprising the eletriptan N-oxide impurity in an amount of about 0.01 area-% to about 0.15 area-%; and wherein the pharmaceutically acceptable salt of eletriptan is a hydrochloride salt, a hydrobromide salt, a hemioxalate salt, a hemisulphate salt, a phosphate salt, a hemifumarate salt, a fumarate salt, a hemisuccinate salt, a succinate salt, a maleate salt, a fumarate salt, a besylate salt, a tosylate salt, or a tartrate salt.

4. Eletriptan of claim 1, having a non-detectable amount of eletriptan N-oxide impurity as measured by HPLC; and wherein the pharmaceutically acceptable salt of eletriptan is eletriptan hydrobromide.

5. (canceled)

6. (canceled)

7. An isolated eletriptan N-oxide, (R)-5-[2-(phenylsulfonyl)ethyl]-3-[(1-methyl-2-pyrrolidinyl-N-oxide)methyl]-1H-indole, of formula A: 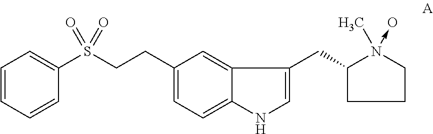
or a pharmaceutically acceptable acid addition salt thereof.

8. A process for preparing highly pure eletriptan or a pharmaceutically acceptable salt thereof of claim 1, comprising:
a) contacting crude eletriptan free base with oxalic acid in a first solvent to produce a first reaction mass containing eletriptan hemioxalate salt;
b) optionally, heating the first reaction mass obtained in step-(a);
c) substantially removing the first solvent from the first reaction mass obtained in step-(a) or step-(b) to produce a solid form of highly pure eletriptan hemioxalate substantially free of eletriptan N-oxide impurity; or
d) isolating the solid form of highly pure eletriptan hemioxalate substantially free of eletriptan N-oxide impurity from the first reaction mass obtained in step-(a) or step-(b) by forcible or spontaneous crystallization;
e) reacting the highly pure eletriptan hemioxalate obtained in step-(c) or step-(d) with an acid and/or a base in a second solvent to produce a second reaction mass containing eletriptan or a pharmaceutically acceptable salt thereof substantially free of eletriptan N-oxide impurity; and
f) isolating and/or recovering the highly pure eletriptan or a pharmaceutically acceptable salt thereof substantially free of eletriptan N-oxide impurity from the second reaction mass.

9. (canceled)

10. The process of claim 8, wherein the first solvent used in step-(a) is selected from the group consisting of water, methanol, ethanol, isopropanol, acetonitrile, and mixtures thereof; and wherein the second solvent used in step-(e) is selected from the group consisting of water, methanol, ethanol, isopropanol, n-butanol, and mixtures thereof.

11. The process of claim 8, wherein the reaction in step-(a) is carried out at a temperature of about 0° C. to the reflux temperature of the solvent used; wherein the reaction mass in step-(b) is heated at a temperature of about 40° C. to the reflux temperature of the solvent used for at least 20 minutes; wherein the removal of solvent in step-(c) is accomplished by substantially complete evaporation of the solvent, concentrating the solution or distillation of solvent, under inert atmosphere; and wherein the reaction in step-(e) is carried out at a temperature of about −25° C. to the reflux temperature of the solvent used.

12. The process of claim 11, wherein the reaction in step-(a) is carried out at a temperature of about 20° C. to about 60° C.; and wherein the reaction in step-(e) is carried out at a temperature of about 20° C. to about 60° C.

13. The process of claim 8, wherein the base used in step-(e) is an organic or inorganic base selected from the group consisting of triethylamine, tributylamine, diisopropylethylamine, diethylamine, tert-butyl amine, N-methylmorpholine, pyridine, 4-(N,N-dimethylamino)pyridine, ammonia, sodium hydroxide, calcium hydroxide, magnesium hydroxide, potassium hydroxide, lithium hydroxide, sodium carbonate, potassium carbonate, lithium carbonate, sodium tert-butoxide, sodium isopropoxide and potassium tert-butoxide; and
wherein the acid used in step-(e) is an organic or inorganic acid selected from the group consisting of hydrochloric acid, hydrobromic acid, hydroiodic acid, acetic acid, fumaric acid, maleic acid, tartaric acid, di-p-toluoyl-L-(+)-tartaric acid, succinic acid, benzenesulfonic acid, toluenesulfonic acid, methanesulfonic acid.

14. (canceled)

15. (canceled)

16. The process of claim 8, wherein the isolation in step-(d) is carried out by stirring the reaction mass at a temperature of about 0° C. to about 30° C.; and wherein the isolation in step-(f) is carried out by stirring the solution at a temperature of about 0° C. to about 30° C. for about 30 minutes to about 20 hours.

17. A process for the preparation of highly pure eletriptan hydrobromide substantially free of eletriptan N-oxide impurity, comprising:
a) providing a solution or suspension of eletriptan hemioxalate in a solvent;
b) combining the solution or suspension obtained in step-(a) with hydrobromic acid; and
c) optionally, heating the reaction mass obtained in step-(b); and
d) isolating and/or recovering highly pure eletriptan hydrobromide substantially free of eletriptan N-oxide impurity from the reaction mass.

18. The process of claim 17, wherein the solvent used in step-(a) is selected from the group consisting of water, methanol, ethanol, n-propanol, isopropanol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, and mixtures thereof; and wherein the hydrobromic acid in step-(b) is used in a molar ratio of about 0.8 to 1.2 moles per 1 mole of the eletriptan hemioxalate.

19. (canceled)

20. (canceled)

21. A process for synthesizing and isolating the eletriptan N-oxide of formula A or a pharmaceutically acceptable acid addition salt thereof of claim 7, comprising:
a) reacting eletriptan base with an oxidizing agent in a suitable solvent to produce a reaction mass containing eletriptan N-oxide; and
b) isolating and/or recovering the eletriptan N-oxide from the reaction mass obtained in step-(a) and optionally converting the eletriptan N-oxide obtained into a pharmaceutically acceptable acid addition salt thereof.

22. The process of claim 21, wherein the oxidizing agent used in step-(a) is selected from the group consisting of m-chloroperbenzoic acid, hydrogen peroxide, sodium hypochlorite and cumene hydrogen peroxide; and wherein the solvent used in step-(a) is selected from the group consisting of acetone, methyl ethyl ketone, methyl isobutyl ketone, methyl tert-butyl ketone, acetonitrile, ethyl acetate, methyl acetate, isopropyl acetate, tert-butyl methyl acetate, ethyl formate, dichloromethane, dichloroethane, chloroform, carbon tetrachloride, tetrahydrofuran, dioxane, diethyl ether, diisopropyl ether, monoglyme, diglyme, n-pentane, n-hexane, n-heptane, cyclohexane, toluene, xylene, and mixtures thereof.

23. The process of claim 22, wherein the oxidizing agent is m-chloroperbenzoic acid; and wherein the solvent is selected from the group consisting of hexane, heptane, cyclohexane, toluene, dichloromethane, and mixtures thereof.

24. The process of claim 21, wherein the reaction in step-(a) is carried out at a temperature of below about 50° C. for at least 30 minutes; wherein the oxidizing agent is used in a molar ratio of about 1 to 4 moles per 1 mole of eletriptan free base; and wherein the isolation in step-(b) is carried by cooling, seeding, partial removal of the solvent from the solution, by adding an anti-solvent to the solution, evaporation, vacuum drying, spray drying, freeze drying, column chromatography, or a combination thereof.

25. The process of claim 24, wherein the reaction in step-(a) is carried out at a temperature of about −20° C. to about 30° C. for about 30 minutes to about 10 hours; and wherein the oxidizing agent is used in a molar ratio of about 2 to 3 moles per 1 mole of eletriptan free base.

26. The eletriptan or a pharmaceutically acceptable salt thereof of claim 1, further comprising one or more pharmaceutically acceptable excipients to form a pharmaceutical composition.

27. (canceled)

28. (canceled)

29. (canceled)

30. The pharmaceutical composition of claim 26, wherein the highly pure eletriptan or a pharmaceutically acceptable salt thereof has a D₉₀ particle size of less than or equal to about 200 microns.

31. The pharmaceutical composition of claim 30, wherein the D₉₀ particle size is in the range between about 1 micron to about 150 microns, or about 10 microns to about 100 microns.

32. (canceled)

33. (canceled)

34. (canceled)
